SEQUENCE LISTING (8 sequences)

SEQ ID NO: 1 - DNA, 1318 bp, E. coli
ttaattttgatgccagccaggttggtcattctcaaatacctcagcctcggggaatattttagtcaatgggataacatatttgaccagatcatgggtaaaaaagtcatccgcagtttcatctaccatccacgcagagttctcatctatgcgtggcacgagataatgaacattcaggcggactattttagatttttgctcaaaatttactatggcagcatagtttttttcccttcgtttatcggcaacatccttttgaatatctacatccaaagctatatgccctacgcctagaccataatccaaaagctggttatctatctctgctaatgtttttgttacatgtcgcgaacgtgcatcgaaagatttttcattgatacaacgccatgttatcaaagaagcaagtttaactttggttatgaaacgtgagtcgcgctcatctggtcgtcccatagcaacaacttggtaattttcatcttccagaggggagcctttgataagacgagcaagtttagtgttgactagtaatgagccgttttcagtgatatcttccctgataagatttaagttggccggtctaatcgttccttttccataatcatcattccattcataaaatacacctttaaagttttttaagtggctaatgatatagttttcaggcgcgtctttaacttcacataaatatgtgacatcagtccatacgttcatctttttggatttgatatgaagttcggcaagatgtgagcgtatatgatgctgtaatatttcctgctttacgtaaggacctttctgaagccttttgcattcgacaaagaaataatcgccatctccaaggcggcaacgaaactccggggtttttgctatccctttttgttctggaatgaactcaacttcataaccttctgatgcatagtttccagcaagaaccaactctaataaggctgtgtctggtaatactgttgtattcttaagcattctgattgcgcgatctctagcgccagtaattctatcgagcgatttagcacacactcctaattgtttaacccatggtattatatttgaagcattagtaacttcgtaagaccttctattatcgataagcgattttgcttgtgcgaaataaccagcaaccacgtcggaaccataccattctgggtcaaactgtttcccgaaattggcagctttttgggttgtagctatgtaaaattgctgtgcttttgctaagcgctggtaaaagtgttgtttgttctcttgagttgatgctaaccactcaagaccagcaataacgtcagaatctagcataacaccgtcattcatt

SEQ ID NO: 2 - DNA, 427 bp, Artificial Sequence
  synthetically prepared oligonucleotide sequence
gctatccctttttgttctggaatgaactcaacttcataaccttctgatgcatagtttccagcaagaaccaactctaataaggctgtgtctggtaatactgttgtattcttaagcattctgattgcgcgatctctagcgccagtaattctatcgagcgatttagcacacactcctaattgtttaacccatggtattatatttgaagcattagtaacttcgtaagaccttctattatcgataagcgattttgcttgtgcgaaataaccagcaaccacgtcggaaccataccattctgggtcaaactgtttcccgaaattggcagctttttgggttgtagctatgtaaaattgctgtgcttttgctaagcgctggtaaaagtgttgtttgttctcttgagttgatgctaaccactcaagaccagcaataa

SEQ ID NO: 3 - DNA, 20 bp, Artificial Sequence
  synthetic primer
gctatccctttttgttctgg

SEQ ID NO: 4 - DNA, 20 bp, Artificial Sequence
  synthetic primer
ttattgctggtcttgagtgg

SEQ ID NO: 5 - DNA, 20 bp, Artificial Sequence
  synthetic primer
cctatccctttttgttctgg

SEQ ID NO: 6 - DNA, 20 bp, Artificial Sequence
  synthetic primer
catccgcagtttcatctacc

SEQ ID NO: 7 - DNA, 20 bp, Artificial Sequence
  synthetic primer
ctcacatcttgccgaacttc

SEQ ID NO: 8 - DNA, 641 bp, Artificial Sequence
  synthetically prepared oligonucleotide sequence
catccgcagtttcatctaccatccacgcagagttctcatctatgcgtggcacgagataatgaacattcaggcggactattttagatttttgctcaaaatttactatggcagcatagtttttttcccttcgtttatcggcaacatccttttgaatatctacatccaaagctatatgccctacgcctagaccataatccaaaagctggttatctatctctgctaatgtttttgttacatgtcgcgaacgtgcatcgaaagatttttcattgatacaacgccatgttatcaaagaagcaagtttaactttggttatgaaacgtgagtcgcgctcatctggtcgtcccatagcaacaacttggtaattttcatcttccagaggggagcctttgataagacgagcaagtttagtgttgactagtaatgagccgttttcagtgatatcttccctgataagatttaagttggccggtctaatcgttccttttccataatcatcattccattcataaaatacacctttaaagttttttaagtggctaatgatatagttttcaggcgcgtctttaacttcacataaatatgtgacatcagtccatacgttcatctttttggatttgatatgaagttcggcaagatgtgag

(19) United States
(12) Patent Application Publication
(10) Pub. No.: US 2006/0105369 A1
(43) Pub. Date: May 18, 2006

(54) Methods and kits for identifying Enterohaemorrhagic Escherichia coli O157:H7 strain

(76) Inventors: Yanmei Xu, Beijing (CN); Jianguo Xu, Beijing (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/257,228

(22) Filed: Oct. 24, 2005

(30) Foreign Application Priority Data
Oct. 22, 2004 (CN) 200410086021.6

(51) Int. Cl. C12Q 1/68; C12P 19/34

(52) U.S. Cl. 435/6; 435/91.2

(57) ABSTRACT

Disclosed is a method for identifying the presence of Enterohaemorrhagic E. coli O157:H7 strain using primers or a probe prepared in accordance with SEQ ID NO: 1 or a sequence homologous thereto. A kit for identifying Enterohaemorrhagic E. coli O157:H7 strain is also disclosed.

### CROSS-REFERENCE TO RELATED APPLICATIONS

This application claims the benefit of Chinese Patent Application No. CN200410086201.6 filed Oct. 22, 2004, entitled the same, which is explicitly incorporated herein by reference in its entirety.

### BACKGROUND OF THE INVENTION

1. Field of the Invention

The present invention relates to a method for identifying the presence of Enterohaemorrhagic Escherichia coli O157:H7 strain in a subject, particularly to a method for identify the presence of Enterohaemorrhagic Escherichia coli O157:H7 strain in a subject with a specific sequence thereof. The present invention also provides a kit for identifying Enterohaemorrhagic Escherichia coli O157:H7 strain.

2. Description of the Related Art

Enterohaemorrhagic Escherichia coli (Enterohaemorrhagic E. coli, EHEC) is a pathogen in gastrointestinal tracts that has recently been found severely harmful to human being. The infection of EHEC O157:H7 has been spreading all over the world since it first caused food poisoning in United States in 1982. Nowadays, EHEC O157:H7 has become one of the most concerned pathogens.

It has been known that EHECs are associated with hemorrhagic colitis (HC), hemolytic uremic syndrome (HUS), and thrombotic thrombocytopenic purpura (TTP) etc. It is estimated by the Center of Disease Control (CDC) of USA that it has caused about 7,000 HC cases per year in the United States in the past several years. Therefore, EHEC O157:H7 has significantly threatened people's health and become a problem of the public health.

A variety of methods for detecting EHEC O157:H7 have been disclosed in the art. These methods are based on various characteristics of the strain that distinguish the strain from others of E. coli, such as not fermenting sorbitol within 24 hours, absence of β-glucuronidase activities, capability of expressing O157 bacterial antigens and H7 flagellum antigens, production of Shiga-like toxins, and encoding hemolysin genes hly and eae. Generally, methods for detecting E. Coli include a selective medium test, a nucleic acid probes test, a serum protein blotting test, a Shiga-like toxin test and the like.

E. coli O157 strains with capability of fermenting sorbitol and β-glucuronidase activities have been discovered. As a result, E. coli O157 strains fail to be specifically identified by those characteristics, like not fermenting sorbitol and absence of β-glucuronidase activities. Moreover, antibodies against O157 bacterial antigens can cross-react with antigens of Salmonella group N, Yersinia and the like. Therefore, E. coli O157 strains cannot be identified by identifying specific antigens thereof.

Because various serotypes of EHECs besides O157:H7, such as O26, O111, O91, O103 and O146, and some strains, such as Shigella, V cholerae and citrus bacillus, comprise Shiga-like toxin genes, it is difficult to differentiate EHEC strains from Enteropathigenic E. coli (EPEC) strains by primers or probes for detecting toxin genes. Also different serotypes of EPECs cannot be determined using such primers or probes.

Furthermore, because Shiga-like toxin genes have different variants, it is difficult to identify all of toxin genes by one pair of primers. EPEC has an LEE (Locus of Enterocyte Effacement) pathogenicity island, of which the eae gene is of a high homology to that of EHEC. A variety of EHEC serotypes such as O26 and O111 contain hemolysin gene hly, which could cause hemorrhagic colitis and hemolytic uremic syndrome. Therefore, the identification of genes eae and hly is not sufficient for specifically identifying Enterohaemorrhagic E. coli O157:H7 strain.

E. coli strains with a bacterial antigen of O157 comprise a variety of flagella antigens, whereas many non-O157 serotype strains, particularly such as E. coli O55:H7 strain, also contain H7 flagellum gene flic. Research has indicated that E. coli O157:H7 strain is evolved from E. coli O55:H7 strain, both having a relatively close genetic relationship. Therefore, similar to the case of serotype O157 strains mentioned above, primers of H7 flagellum genes can be used limitedly to assist the identification of Escherichia coli O157:H7 strain.

There has been no report on the application of specific primers or probes for identification of EHEC O157:H7 strain. The whole genome size of EHEC O157:H7 strain is about 5.5 Mb. It is a pending technical issue for finding a specific sequence that differs from any of other strains and can be used to specifically identify strain itself. Accordingly, methods for specifically detecting and identifying EHCC O157:H7 have not been disclosed in the art.

### SUMMARY OF THE INVENTION

Therefore, there is a need for a specific sequence for identifying EHEC O157:H7 strain and a method for rapidly and specifically detecting and identifying EHEC O157:H7 strain with the specific sequence.

Preferred embodiments provide a method for identifying the presence of Enterohaemorrhagic E. coli O157:H7 strain in a subject, comprising:

providing a sample obtained from the subject;

providing primers that bind to SEQ ID NO:1 or its complement or a sequence homologous to either of the foregoing;

contacting the sample with the primers;

amplifying primer sequences to produce an amplified product, if Enterohaemorrhagic E. coli O157:H7 strain is present; and

detecting the amplified product, if Enterohaemorrhagic E. coli O157:H7 strain is present, thereby indicating presence of Enterohaemorrhagic E. coli O157:H7 strain in the sample.

Preferred embodiments provide a method for identifying the presence of Enterohaemorrhagic E. coli O157:H7 strain in a subject, comprising:

providing a sample obtained from the subject;

providing a probe that binds to SEQ ID NO:1 or its complement or a sequence homologous to either of the foregoing;

contacting the sample with the probe;

identifying binding of the probe to the sample, thereby indicating presence of Enterohaemorrhagic E. coli O157:H7 strain in the sample.

Preferred embodiments provide a kit for identifying Enterohaemorrhagic Escherichia coli O157:H7 strain comprising:

a primer or a probe that bind to SEQ ID NO:1 or its complement or a sequence homologous to either of the foregoing; and

instructional materials.

Preferred embodiments provide a primer that bind to SEQ ID NO:1 or its complement or a sequence homologous to either of the foregoing.

Preferred embodiments provide a probe prepared that bind to SEQ ID NO:1 or its complement or a sequence homologous to either of the foregoing.

### BRIEF DESCRIPTION OF THE DRAWINGS

FIG. 1 shows the PCR results of some tested strains with the primers of SEQ ID NO: 3 and SEQ ID NO: 4 according to the preferred embodiments, in which a band about 427 bp was observed in the lane of Enterohaemorrhagic E. coli O157:H7 strain and the lane of a positive control respectively, which coincides with the predicted DNA length to be amplified by the designed primers, while the lanes for other strains were negative.

FIG. 2 shows the results of Southern blot hybridization between chromosome of some strains and the labeled probe generated from PCR products of the primers of SEQ ID NO: 3 and SEQ ID NO: 4 according to the preferred embodiments, in which a positive band was obtained in both the lanes of Enterohaemorrhagic E. coli O157:H7 strain and the positive control, while other strains were negative.

FIG. 3 shows the PCR results of some strains by the primers of SEQ ID NO: 5 and SEQ ID NO: 4 according to the preferred embodiments, in which a band about 427 bp was obtained in the lanes of Enterohaemorrhagic E. coli O157:H7 strain and the positive control, which coincides with the predicted length of a product to be generated by the designed primers, while other strains were negative.

FIG. 4 shows the PCR result of some strains by the primers of SEQ ID NO: 6 and SEQ ID NO: 7 according to the preferred embodiments, in which a band of about 641 bp was obtained in the lanes of Enterohaemorrhagic E. coli O157:H7 strain and a positive control, which coincides with the predicted length of a product to be generated by the designed primers, while other strains were negative.

### DETAILED DESCRIPTION OF THE PREFERRED EMBODIMENT

The sequence of SEQ ID NO:1 of Enterohaemorrhagic E. coli O157:H7 strain in the preferred embodiments is shown in the Sequence Listing, which is derived from Genebank with the gene identification number of GI:12519288.

The term of “homologous” used herein means a variety of sequences having at least about 80% homology to each other, preferably at least about 85% homology to each other, more preferably at least about 90% homology to each other, and most preferably at least about 95% homology to each other. A skilled person in the art can obtain sequences homologous to a specific sequence like SEQ ID NO: 1 according to known techniques.

Preferred embodiments provide methods to design suitable primers or probes that bind to SEQ ID NO: 1 that are suitable for detecting a nucleic acid sequence fragment of SEQ ID NO: 1.

Preferred embodiments provide a method for identifying the presence of Enterohaemorrhagic E. coli O157:H7 strain in a subject, comprising: providing a sample obtained from the subject; providing primers that bind to SEQ ID NO:1 or its complement or a sequence homologous to either of the foregoing; contacting the sample with the primers; amplifying primer sequences to produce an amplified product, if Enterohaemorrhagic E. coli O157:H7 strain is present; and detecting the amplified product, if Enterohaemorrhagic E. coli O157:H7 strain is present, thereby indicating presence of Enterohaemorrhagic E. coli O157:H7 strain in the sample.

Preferred embodiments provide a method for identifying the presence of Enterohaemorrhagic E. coli O157:H7 strain in a subject, comprising: providing a sample obtained from the subject; providing a probe that binds to SEQ ID NO:1 or its complement or a sequence homologous to either of the foregoing; contacting the sample with the probe; identifying binding of the probe to the sample, thereby indicating presence of Enterohaemorrhagic E. coli O157:H7 strain in the sample.

Preferred embodiments provide a kit for identifying Enterohaemorrhagic Escherichia coli O157:H7 strain comprising: a primer or a probe that bind to SEQ ID NO:1 or its complement or a sequence homologous to either of the foregoing; and instructional materials.

Preferred embodiments provide a primer that bind to SEQ ID NO:1 or its complement or a sequence homologous to either of the foregoing.

Preferred embodiments provide a probe prepared that bind to SEQ ID NO:1 or its complement or a sequence homologous to either of the foregoing.

Examples of primers suitably used in the preferred embodiments include those having sequences selected from the group consisting of SEQ ID NO: 3, SEQ ID NO: 4, SEQ ID NO: 6, SEQ ID NO: 7, and sequences homologous thereto. A probe suitably used in the preferred embodiments can have a sequence selected from the group consisting of SEQ ID NO:1, SEQ ID NO:2, SEQ ID NO:8, SEQ ID NO:3, SEQ ID NO:4, SEQ ID NO:6, SEQ ID NO:7, and a sequence homologous thereto.

It will be appreciated by those skilled in the art that addition, deletion, and variation to the primers or probe described herein may be further made to obtain sequences homologous thereto. For example, SEQ ID NO: 3 can be modified to 5′-CCT ATC CCT TTT TGT TCT GG-3′ of SEQ ID NO: 5.

In other embodiments, the detecting of gene sequences in a subject may be conducted by a method selected from the group consisting of PCR amplification, Southern blotting hybridization, in situ hybridization and dot hybridization.

In a preferred embodiment, a sample from a subject is detected by PCR amplification with primers SEQ ID NO: 3 and SEQ ID NO: 4, to obtain a nucleic acid sequence of SEQ ID NO: 2, i.e. a fragment of SEQ ID NO: 1. Thus, the sample is identified as or comprising Enterohaemorrhagic Escherichia coli O157:H7 strain. The sequences of SEQ ID NO: 3, SEQ ID NO: 4 and SEQ ID NO: 2 are shown in the Sequence Listing.

In another embodiment, a sample from a subject is detected by PCR amplification with the primers of SEQ ID NO: 6 and SEQ ID NO: 7, to obtain a nucleic acid sequence of SEQ ID NO: 8, i.e. a fragment of the sequence SEQ ID NO: 1. Thus, the sample is identified as or comprising Enterohaemorrhagic Escherichia coli O157:H7 strain. The sequences of SEQ ID NO: 6, SEQ ID NO: 7 and SEQ ID NO: 8 are shown in the Sequence Listing.

In certain embodiments, PCR amplification conditions that can be used include: a final concentration of the primers of SEQ ID NO: 3 and SEQ ID NO: 4 or SEQ ID NO: 6 and SEQ ID NO: 7 ranging from about 0.25 μM to about 0.5 μM; an annealing temperature of PCR amplification of the SEQ ID NO: 3 and SEQ ID NO: 4 or SEQ ID NO: 6 and SEQ ID NO: 7 ranging from about 50° C. to about 54° C.; a final concentration of Taq DNA polymerase ranging from about 0.05 U/μl to about 0.075 U/μl.

An artisan may readily determine PCR amplification conditions by specific primers and other conditions. Any of the large number of other amplification methods can be used, for example, different types of PCR amplification, nucleic acid sequence-based amplification (NASBA), rolling circle amplification, Lex, Transcription Mediated Amplification (TMA), ligase chain reaction, ligase-dependent PCR, strand displacement amplification, branched DNA signal amplification, and hybridization signal amplification method.

A subject providing a sample may be a food, an animal, a human, and the like, which is suspected to be contaminated by Enterohaemorrhagic E. colis, or the strain itself.

A sample can be a solid or liquid obtained from the subject. Examples of samples include, but are not limited to, serum, blood, feces, tissue, and the like.

The preferred embodiments also provide a kit for identifying Enterohaemorrhagic E. coli O157:H7 strain, which can include primers or probes defined herein. These kits can be PCR amplification kits, Southern blotting hybridization kits, in situ hybridization kits, and dot hybridization kits, and the like.

In one embodiment, the PCR amplification kit comprises primers SEQ ID NO: 3 and SEQ ID NO: 4, or SEQ ID NO: 6 and SEQ ID NO: 7, and other components well known by artisan in the art, such as a Taq polymerase, reaction buffer, dNTP, water, and the like.

The disclosure below is of specific examples setting forth preferred methods for making preferred compounds. These examples are not intended to limit the scope, but rather to exemplify preferred embodiments.

### EXAMPLE 1

### Detecting of Enterohaemorrhagic E. Coli O157:H7 Strain

(1) Experimental Bacterial Strains

234 strains were selected in this Example, which are:

7 international or national reference strains of Enterovirulent E. coli;

6 strains of EPEC;

8 strains of Enterotoxingenic E. coli (ETEC);

1 strain of Uropathogenic E. coli (UPEC);

1 strain of EHEC O26;

1 strain of EHEC O111;

46 strains of Shigella;

4 strains of Yersinia;

4 strains of Klebsiella pneumoniae;

12 strains of Salmonella (including groups A, B, C, D, E, F and I);

10 strains of C. freundii;

2 strains of Morganella morganii;

1 strain of V. cholerae;

1 strain of Proteus;

1 stain of Enterobacteriaceae;

1 strain of Providencia;

1 strain of H. alve; and

127 strains of E. coli O157 isolated from patients or animals, of which 62 were E. coli O157:H7 strains and 65 were non-E. coli O157:H7 strains.

An EHEC O157:H7 EDL933 strain sequenced in the United States was used as a positive control, and an E. coli MG1655 strain was used as a negative control.

(2) Templates for PCR Amplification

A selected single colony was applied on an LB plate and cultured at 37° C. overnight. Then, a proper amount of the colony was scratched into a 1.5 ml microcentrifuge tube, which was suspended in water. The tube was boiled in boiling water for 10 min, and then centrifugated at 12,000 rpm for 10 min. The supernatant contained a template DNA, which was stored at −20° C. for use.

(3) PCR Amplification

To a 0.5 ml centrifuge tube were added the following regents: 11.7 μl water, 1.2 μl MgCl₂, 2 μl 10× buffer, primers SEQ ID NO: 3 and SEQ ID NO: 4 each at a final concentration of 0.5 μM, dNTP each at a final concentration of 0.2 mM, Taq DNA Polymerase at a final concentration of 0.075 U/μl, and 1 μl template. The reaction system was kept at a final volume of 20 μl.

The resultant was mixed uniformly. PCR amplification was performed as follows: initially denaturizing at 94° C. for 5 minutes, followed by 30 cycles of the amplification (94° C., 1 min; 54° C., 1 min; 72° C., 1 min), and finally extending at 72° C. for 8 minutes.

(4) Detecting of PCR Amplification Products

PCR amplification products were detected by conventional agarose gel electrophoresis. See Molecular Cloning, 2nd edition.

(5) Sequencing of PCR Amplification Products

PCR amplification products were purified by a purification kit and the resultant then was sequenced.

(6) Results

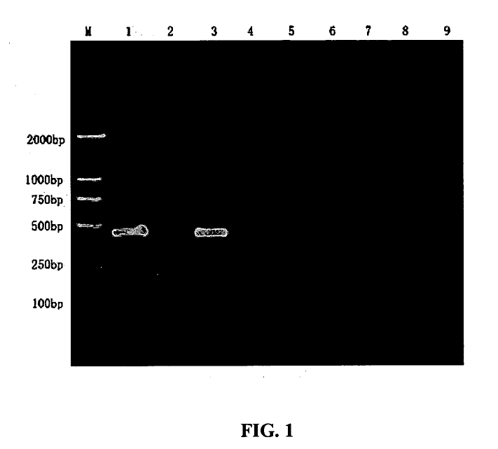

FIG. 1 shows the result of PCR amplification products by electrophoresis. Lane M represented marker DL2000; lane 1 represented the positive control EHEC O157:H7 EDL933 strain; lane 2 represented the negative control E. coli MG1655 strain; lane 3 to lane 9 respectively represented an EHEC O157:H7 strain from the 62 EHEC O157:H7 strains, E. coli O157:H42, EPEC2348/69, Shigella strain 301, Yersinia enterocolitica O:3, Salmonella 50001-24, and Klebsiella pneumoniae.

The result showed that the products of PCR amplification for both the positive control EHEC O157:H7 EDL933 strain and an EHEC O157:H7 strain from the 62 EHEC O157:H7 strains were positive, while others were negative.

No nucleotide mutation or loss was found in the alignment between the sequencing results and the specific sequence SEQ ID NO: 1, and therefore, the strain was identified as Enterohaemorrhagic Escherichia coli O157:H7 strain.

(7) Conclusion

The result showed that primer SEQ ID NO: 3 and primer SEQ ID NO: 4 designed upon SEQ ID NO: 1 and the homologous sequence SEQ ID NO: 2 can be used for identifying EHEC O157:H7 strains and at least reach 48CFU/PCR in a 20 μl reaction volume.

### EXAMPLE 2

### Southern Blotting Hybridization for Detecting Enterohaemorrhagic E. coli O157:H7 STRAIN

(1) Preparation of Probes

A probe was obtained by labeling gel-retrieved PCR amplification products of primers SEQ ID NO: 3 and SEQ ID NO: 4 with DIG DNA Labeling and Detection Kit (Roche Diagnostics Corporation).

(2) Southern Blotting Hybridization

As described by Sambrook et al. 1989, Southern blotting analysis was performed as follows:

1) digesting bacterium chromosomes by a restriction enzyme EcoR V overnight;

2) separating the digested product by conventional electrophoresis;

3) transferring onto membranes and treating the membrane; and

4) pre-hybridization and detecting.

Step 3) is described as follows:

Electrophoretic gel was washed by distilled water to remove excessive EB thereon and the redundant gel was removed. The top left was cut as a sign. The gel was soaked in a denaturation solution (0.5M NaOH, 1.5M NaCl) for 45 minutes and kept shaking, then rinsed briefly with distilled water.

The gel was neutralized twice with a solution (1.5M NH₄Ac, 0.02M NaOH; or 0.5M Tris.HCl, 3M NaCl, pH7.5), each for 15 minutes, and rinsed with distilled water.

A nitrocellulose membrane was prepared by soaking a conventional membrane in distilled water and soaking for at least 5 minutes in 20SSC (3M NaCl, 300 mM Sodium Citrate, pH7.0). Two pieces of filter paper with the same size as the membrane were soaked in a 2×SSC solution (by diluting 20×SSC 10 times with distilled water).

The above gel was turned over (loading wells facing down) and placed on a support coated by a piece of the filter paper rounded by a piece of plastic paper to avoid the short circuit. The membrane was then placed on the gel, and air bubbles were driven away. The filter paper was placed on the membrane, and a blotter having a smaller size than the filter paper was placed on the filter paper. It took 8 to 24 hours for the membrane transfer process. After that, the membrane was soaked in a 6×SSC solution (by diluting 20×SSC 3.33 times with distilled water) for 5 min to remove agarose residues sticking to the membrane. The membrane was dried on a filter paper for 30 minutes, and then followed by fixing at 80° C. for 2 hours.

Step 4) is carried out as follows.

The membrane prepared in step 3) was put into a bag with a pre-hybridization solution (5×SSC, 0.1 of sodium dodecyl sarcosinate, 0.02% sodium dodecyl sulphonate, and 2% blocking solution). Then the bag was sealed and incubated in water bath at 68° C. for 2 hours.

The probe was added into a small volume of the pre-hybridization solution. The resultant was boiled for 10 minutes, and laid on ice immediately. An amount of the pre-hybridization solution was added to render the concentration of the probe ranging from 5 to 25 ng/ml. The hybridization solution was added into the bag.

The bag was incubated in water bath at 68° C. for 16 hours.

The membrane was then washed twice with Wash Solution I (2×SSC, 0.1% SDS), 5 min each; and twice at 68° C. with Wash Solution II (0.1×SSC, 0.1% SDS), 15 minutes each.

The membrane successively washed with Buffer I (0.1M maleic acid, 0.15M NaCl, 50 mM MgCl₂, adjusted to pH7.5 with NaOH) for 1 minute, and Buffer II (a 10× bolcking solution, made by diluting a solid blocking agent in Buffer I, with a final concentration of the blocking agent 1% (v/v) in the blocking solution) for 30 to 60 minutes. The membrane was then incubated with Buffer II plus an antibody (1:10,000) for 30 minutes at room temperature, and washed twice with Buffer I, each for 15 minutes.

Then the membrane was neutralized with Buffer III (100 mM Tris.HCl, 100 mM NaCl, 50 mM MgCl₂, pH9.5) 2 to 5 minutes and revealed with a color-developing agent of 200 μl NBT/BCIP in 10 ml Buffer III.

The above mentioned solid blocking agent, antibody, and color-developing agent were available from DIG DNA Labeling and Detection Kit.

(3) Southern Blotting Hybridization

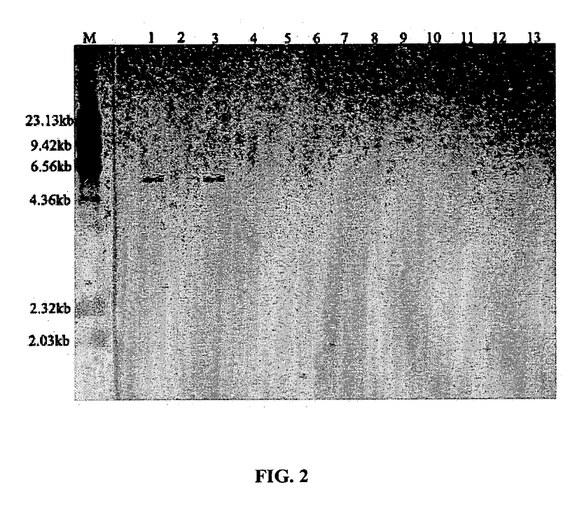

FIG. 2 showed the result of Southern blotting hybridization, lane M representing λDNA HindIII marker; lane 1 representing the positive control of EHEC O157:H7 EDL933 strain; lane 2 representing the negative control of E. coli MG1655 strain; and lane 3 to lane 13 representing an EHEC O157:H7 strain from the 62 EHEC O157:H7 strains, an E. coli O157:H42 strain, EPEC2348/69, Shigella strain 301, Yersinia enterocolitica O:3, Salmonella 50001-24, Klebsiella pneumoniae, EIEC 8401, ETEC 10407, EAggEC O42, and UPEC CFT073, respectively.

The result showed that only the hybridization for EHEC O157:H7 EDL933 strain as the positive control and an EHEC O157:H7 strain from the 62 EHEC O157:H7 strains in lane 3 were observed (positive) and others were negative.

(4) Conclusion

The result suggested that sequence SEQ ID NO: 2 could be used as probes for identifying EHEC O157:H7 strain by Southern blotting hybridization, and the PCR amplification products belonged to the specific sequence of EHEC O157:H7 strain.

### EXAMPLE 3

### Specificity of Primers SEQ ID NO: 5 and SEQ ID NO: 4

(1) Experimental Bacterial Strains

232 bacterial strains were selected in this experiment:

7 international or national reference strains of Enterovirulent E. coli;

6 strains of EPEC;

8 strains of Enterotoxingenic E. coli (ETEC);

1 strain of Uropathogenic E. coli (UPEC);

1 strain of EHEC O26;

1 strain of EHEC O111;

46 strains of Shigella;

4 strains of Yersinia

4 strains of Klebsiella pneumoniae;

11 strains of Salmonella (including groups A, B, C, D, E, F and I);

9 strains of C. freundii;

2 strain of Morganella morganii;

1 strain of V. cholerae;

1 strain of Proteus;

1 strain of Enterobacteriaceae;

1 strain of Providencia;

1 strain of H. alve; and

127 strains of E. coli O157 isolated from patients or animals, of which 62 were E. coli O157:H7 strains and 65 were other H serotypes of E. coli O157.

An EHEC O157:H7 EDL933 strain sequenced in the United States was designed as a positive control strain in the experiment and E. coli MG1655 was designed as a negative control strain.

(2) Methodologv

Some nucleotides in SEQ ID NO: 3 were changed to obtain a primer of SEQ ID NO: 5, i.e. 5′-CCT ATC CCT TTT TGT TCT GG-3′, which was paired with the primer of SEQ ID NO: 4, so as to perform PCR amplification according to Example 1.

PCR amplification products were purified by a post-electrophoresis gel cutting purification kit, and sequenced according to Example 1. The sequencing result was aligned with the specific sequence of SEQ ID NO: 1.

(3) Results

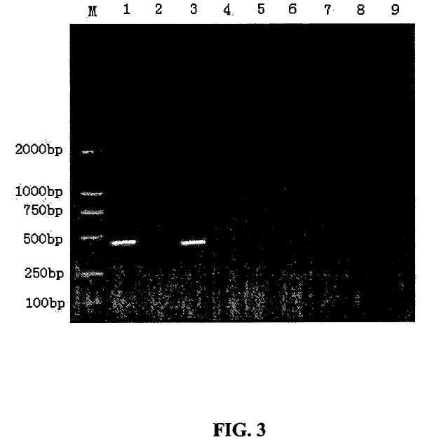

FIG. 3 showed the result of the PCR amplifying products with primer SEQ ID NO: 5 and primer SEQ ID NO: 4, in which lane M represented marker DL2000; lane 1 represented the positive control of EHEC O157:H7 EDL933 strain; lane 2 represented the negative control of E. coli MG1655 strain; lane 3 to lane 9 represented an EHEC O157:H7 strain from the 62 EHEC O157:H7 strains, an E. coli O157:H42, EPEC2348/69, the Shigella strain 301, Yersinia enterocolitica O:3, Salmonella 50001-24, and Klebsiella pneumoniae, respectively.

The result showed that only the hybridization for EHEC O157:H7 EDL933 strain as the positive control and the strain from the 62 EHEC O157:H7 strains were observed (positive), with the same length as the amplification sections of SEQ ID NO:3 and SEQ ID NO:4; and others PCR amplification products were negative.

No nucleotide mutation or deletion was found in the alignment between the above sequence and the specific sequence of SEQ ID NO: 1, which was identified as Enterohaemorrhagic Escherichia coli O157:H7 strain.

(4) Conclusion

The result showed that primer SEQ ID NO: 5 had the identical specificity to the primer SEQ ID NO: 4, and could be used for identifying EHEC O157:H7 strain.

### EXAMPLE 4

### Identifying Enterohaemorrhagic E. coli O157:H7 Strain by PCR

(1) Experimental Bacterial Strains

131 strains were selected in this experiment:

7 international or national strains of Enterovirulent E. coli;

1 strain of EHEC O26;

1 strain of EHEC O111;

45 strains of Shigella;

3 strains of Yersinia

4 strains of Klebsiella pneumoniae;

10 strains of Salmonella (including groups A, B, C, D, E, F and I);

7 strains of C. freundii;

2 strains of Morganella morganii;

1 strain of V. cholerae;

1 strain of Proteus;

1 strain of Enterobacteriaceae;

1 strain of Providencia;

1 strain of H. alve; and

46 strains of E. coli O157 isolated from patients or animals, of which 19 were E. coli O157:H7 strains and 27 were other H serotypes of E. coli O157.

An EHEC O157:H7 EDL933 strain sequenced in the United States was designed as a positive control strain in the experiment and E. coli MG1655 was designed as a negative control strain.

(2) Methodology

A pair of primers of SEQ ID NO: 6 and SEQ ID NO: 7 were designed based on the part of 5′ terminal of SEQ ID NO: 1 which was 5′-CAT CCG CAG TTT CAT CTA CC-3′ and 5′-CTC ACA TCT TGC CGA ACT TC-3 respectively.

The PCR amplification was performed at conditions: a initial denaturation at 94° C. for 5 minutes, followed by 30 cycles of amplification (94° C., 1 min; 54° C., 1 min; 72° C., 1 min) and a final extension at 72° C. for 8 minutes. A sequence of SEQ ID NO: 8 was obtained with a length of 641 bp.

The PCR amplification products were purified by a purifying kit after electrophoresis, and sequenced in the same manner as in Example 1. The sequencing result was aligned with the specific sequence of SEQ ID NO: 1.

(3) Results

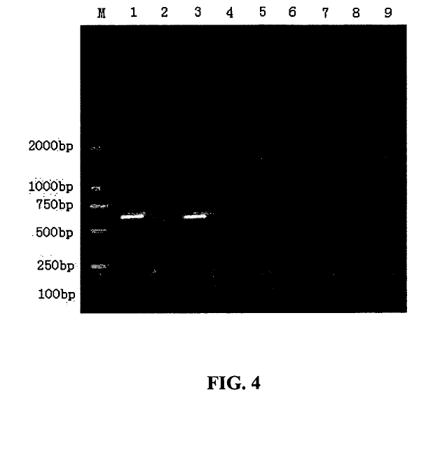

FIG. 4 showed the result of PCR amplification products with primer SEQ ID NO: 6 and primer SEQ ID NO: 7, lane M representing marker DL2000; lane 1 representing the positive control of EHEC O157:H7 EDL933 strain; lane 2 representing the negative control of E. coli MG1655 strain; and lane 3 to lane 9 representing an EHEC O157:H7 strain from the 19 EHEC O157:H7 strains, an E. coli O157:H42 strain, EPEC2348/69, Shigella strain 301, Yersinia enterocolitica O:3, Salmonella 50001-24, and Klebsiella pneumoniae, respectively.

The result showed that only PCR amplification products of the positive control EHEC O157:H7 EDL933 strain and EHEC O157:H7 from the strain were observed positive and other PCR amplification products were negative.

No nucleotide mutation or deletion was found in the alignment between the above sequence and the specific sequence of SEQ ID NO: 1, which was identified as Enterohaemorrhagic E. coli O157:H7 strain.

(4) Conclusion

The result showed that primers designed on SEQ ID NO: 1 and a homologous sequence thereof SEQ ID NO: 8 could be used for identifying EHEC O157:H7 strain.

It is understood that embodiments and examples provided herein as well as the description thereof are only for illustrating the present invention, and various modifications or changes in light thereof will be suggested to persons skilled in the art and are to be included within the spirit and purview of this application and scope of the appended claims.

What is claimed is:

1. A method for identifying the presence of Enterohaemorrhagic E. coli O157:H7 strain in a subject, comprising:
providing a sample obtained from the subject;
providing primers that bind to SEQ ID NO: 1 or its complement or a sequence homologous to either of the foregoing;
contacting the sample with the primers;
amplifying primer sequences to produce an amplified product, if Enterohaemorrhagic E. coli O157:H7 strain is present; and
detecting the amplified product, if Enterohaemorrhagic E. coli O157:H7 strain is present, thereby indicating presence of Enterohaemorrhagic E. coli O157:H7 strain in the sample.

2. The method of claim 1, wherein the primers are selected from the group consisting of SEQ ID NO:3, SEQ ID NO:4, SEQ ID NO:6, SEQ ID NO:7, and a sequence homologous thereto.

3. The method of claim 2, wherein amplification is PCR amplification.

4. The method of claim 3, wherein the PCR is performed with the primers at a final concentration ranging from about 0.25 μM to about 0.5 μM

5. The method of claim 3, wherein the PCR is performed with an annealing temperature of PCR amplification ranging from about 50° C. to about 54° C.

6. The method of claim 3, wherein the PCR is performed with a Taq DNA polymerase at a final concentration ranging from about 0.05 U/μl to about 0.075 U/μl.

7. A method for identifying the presence of Enterohaemorrhagic E. coli O157:H7 strain in a subject, comprising:
providing a sample obtained from the subject;
providing a probe that binds to SEQ ID NO:1 or its complement or a sequence homologous to either of the foregoing;
contacting the sample with the probe;
identifying binding of the probe to the sample, thereby indicating presence of Enterohaemorrhagic E. coli O157:H7 strain in the sample.

8. The method according to claim 7, further comprising providing a second probe that binds to SEQ ID NO:1 or its complement or a sequence homologous to either of the foregoing.

9. The method of claim 8, wherein the probes are primers for amplification, and wherein the identifying step comprises amplifying the primer sequences to produce an amplified product.

10. The method of claim 7, wherein the probe is selected from the group consisting of SEQ ID NO:1, SEQ ID NO:2, SEQ ID NO:8, SEQ ID NO:3, SEQ ID NO:4, SEQ ID NO:6, SEQ ID NO:7, and a sequence homologous thereto.

11. The method of claim 7, wherein the identifying step comprises Southern blotting hybridization, in situ hybridization, or dot hybridization.

12. A kit for identifying Enterohaemorrhagic Escherichia coli O157:H7 strain comprising:
a primer or a probe that bind to SEQ ID NO:1 or its complement or a sequence homologous to either of the foregoing; and
instructional materials.

13. The kit of claim 12, wherein the primer is selected from the group consisting of SEQ ID NO: 3, SEQ ID NO: 4, SEQ ID NO: 6, SEQ ID NO: 7, and a sequence homologous thereto.

14. The kit of claim 12, wherein the probe is selected from the group consisting of SEQ ID NO:1, SEQ ID NO:2, SEQ ID NO:8, SEQ ID NO:3, SEQ ID NO:4, SEQ ID NO:6, SEQ ID NO:7, and a sequence homologous thereto.

15. A primer that bind to SEQ ID NO:1 or its complement or a sequence homologous to either of the foregoing.

16. The primer of claim 15, wherein the primer has a sequence selected from the group consisting of SEQ ID NO:3, SEQ ID NO:4, SEQ ID NO:6, SEQ ID NO:7, and a sequence homologous thereto.

17. A probe prepared that bind to SEQ ID NO:1 or its complement or a sequence homologous to either of the foregoing.

18. The probe of claim 17, wherein the probe has a sequence selected from the group consisting of SEQ ID NO:1, SEQ ID NO:2, SEQ ID NO:8, SEQ ID NO:3, SEQ ID NO:4, SEQ ID NO:6, SEQ ID NO:7, and a sequence homologous thereto.
